(19) United States
(12) Patent Application Publication
(10) Pub. No.: US 2016/0243381 A1
(43) Pub. Date: Aug. 25, 2016

(54) SYSTEMS AND TECHNIQUES FOR ULTRASOUND NEUROPROTECTION

(71) Applicant: Medtronic, Inc., Minneapolis, MN (US)

(72) Inventors: Jamu K. Alford, Ham Lake, MN (US); Steven M. Goetz, North Oaks, MN (US)

(21) Appl. No.: 14/627,843

(22) Filed: Feb. 20, 2015

(51) Int. Cl. A61N 7/00

(52) U.S. Cl. CPC A61N 7/00; A61N 2007/0021

(57) ABSTRACT

The disclosure describes devices, systems, and techniques for reducing neural degeneration within a brain of a patient. In one example, a method includes delivering, via one or more ultrasound transducers, ultrasound energy focused to a targeted region of the brain of the patient according to ultrasound parameters. The ultrasound parameters are selected to generate ultrasound energy that reduces or prevents neural degeneration within at least a portion, such as a selected region, of the brain associated with the targeted region of the brain. The targeted region of the brain may include at least a portion of the selected region of the brain and/or neurons that affect different neurons within the selected region of the brain.

### TECHNICAL FIELD

The disclosure relates to medical therapies and, more particularly, ultrasound delivery.

### BACKGROUND

Neurodegenerative diseases can occur in older adults and may result in cognitive impairment of brain function, motor dysfunction, or even death. Such diseases may include Parkinson's disease, Alzheimer's disease, amyotrophic lateral sclerosis (ALS), Huntington's disease, and others. A clinician may treat a patient with a neurodegenerative disease using one or more therapies. Oral medication may be prescribed for some patients. Patients may also or alternatively be treated using drug delivery therapy and/or electrical stimulation therapy. Electrical stimulation therapy may include deep brain stimulation (DBS), although other types of electrical stimulation therapy may be employed for some patients. Typically, patients are not treated with DBS until after other, less invasive, treatments are not efficacious.

### SUMMARY

In general, the disclosure is directed to techniques and/or systems for reducing degeneration of neurons of a brain of a patient. For example, at early stages of a neurodegenerative disease, a system may be configured to deliver ultrasound energy to a targeted region of the brain of the patient to reduce or prevent the degeneration of neurons in a selected region of the brain that may be similar or different from the targeted region. The system may include one or more ultrasound transducers placed on an exterior surface of the patient's head and configured to deliver the ultrasound energy focused to the targeted region. The ultrasound energy may be defined by a set of ultrasound parameters selected to affect the selected region of the brain corresponding to the neurodegenerative disease. The ultrasound energy may stimulate the neurons within the selected region of the brain associated with the targeted region of the brain receiving the ultrasound energy. The targeted region may be completely separate or distinct from or at least partially include the selected region. This stimulation resulting from the delivery of ultrasound energy can provide neuroprotective effects that may reduce, halt, or even reverse the degeneration of neurons in the selected region of the brain that may otherwise occur from the neurogenerative disease.

In one aspect, the disclosure is directed to a method for reducing or preventing neural degeneration within a brain of a patient, wherein the method includes delivering, via one or more ultrasound transducers, ultrasound energy focused to a targeted region of the brain of the patient according to ultrasound parameters, wherein the ultrasound parameters are selected to generate ultrasound energy that reduces or prevents neural degeneration within at least a portion of the brain.

In another aspect, the disclosure is directed to a system for reducing or preventing neural degeneration within a brain of a patient, wherein the system includes an ultrasound module configured to deliver, via one or more ultrasound transducers, ultrasound energy focused to a targeted region of the brain of the patient and a processor configured to control the ultrasound module to deliver the ultrasound energy to the targeted region according to ultrasound parameters, wherein the ultrasound parameters are selected to generate the ultrasound energy that reduces or prevents neural degeneration within at least a portion of the brain.

In a further aspect, the disclosure is directed to a system for reducing or preventing neural degeneration within a brain of a patient, wherein the system includes means for delivering ultrasound energy focused to a targeted region of the brain of the patient and means for controlling the means for delivering ultrasound energy to deliver the ultrasound energy to the targeted region according to ultrasound parameters, wherein the ultrasound parameters are selected to generate the ultrasound energy that reduces or prevents neural degeneration within at least a portion of the brain.

The details of one or more example are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example system that delivers ultrasound energy to a targeted region of a brain of a patient to reduce neural degeneration within a selected region of the brain, according to one or more aspects disclosed herein.

FIG. 2 is a conceptual diagram illustrating an example wearable device that includes an array of ultrasound transducers that deliver ultrasound energy to a targeted region of the brain.

FIG. 3 is a conceptual diagram illustrating example ultrasound transducers that can be used to focus ultrasound energy to a targeted region of a brain.

FIG. 4 is a schematic diagram of example regions and circuits within a brain of a patient.

FIG. 5 is a block diagram illustrating an example configuration of a controller device which may be utilized in the system of FIGS. 1 and 2.

FIG. 6 is a flow diagram that illustrates an example process for determining a set of ultrasound parameters that at least partially define ultrasound energy deliverable to a targeted region of a brain of a patient.

FIG. 7 is a flow diagram that illustrates an example process for delivering ultrasound energy focused to a targeted region of a brain of a patient to reduce neural degeneration within a selected region of the brain.

### DETAILED DESCRIPTION

The disclosure is directed to techniques and systems for reducing degeneration of neurons within of a brain of a patient. Various neurodegenerative diseases (e.g., Parkinson's disease, Alzheimer's disease, amyotrophic lateral sclerosis (ALS), Huntington's disease, dystonia, tremor, dyskinesia, bradykinesia, dementia, or chronic pain) occur in the adult population and can include symptoms such as motor dysfunction and/or cognitive impairment. The cause of these symptoms typically includes the degeneration of neural networks within one or more regions of the brain over time. Degeneration of neurons may be caused by dopamine depleting neurotoxins, chemicals, viruses, tumors, and/or one or more physiological events. Neurons may degenerate over weeks, months, or years resulting in increased, or more frequent, symptoms.

Deep brain electrical stimulation (DBS) directed to one or more regions of the brain may be an effective treatment of motor dysfunction and/or cognitive impairment resulting from a neurogenerative disease. For example, DBS may alleviate tremors, bradykinesia, and speech problems experienced by a patient diagnosed with Parkinson's disease. In addition, DBS may even slow the progression of a neurodegenerative disease. The electrical stimulation from DBS may protect the nigrostratial system, for example, from dopamine depleting neurotoxins that would otherwise contribute to the degeneration of the neurons. However, DBS may not be an appropriate preventative therapy for patients recently diagnosed with, or at risk for, neurodegenerative diseases. Since DBS is an invasive therapy that requires electrodes implanted within the brain of the patient, DBS is typically reserved for patients in whom the neurogenerative disease has progressed to a stage at which less invasive therapies (e.g., oral medications or other drug therapies) no longer alleviate symptoms. At this later stage in the progression of the disease, when significant neuron loss has already occurred—sometimes over five years from when the first symptoms were identified—the benefits from protecting remaining neurons may be minimal.

As described herein, various techniques and systems may be used to non-invasively reduce or prevent degeneration of neurons in the brain of the patient. For example, a system may include one or more ultrasound transducers placed on an exterior surface of the patient's head (e.g., in contact with the skin of the head) and configured to deliver the ultrasound energy focused to a targeted region of the brain. The ultrasound transducers may take the form of an array of ultrasound transducers positioned on the head of the patient in a configuration selected to focus the ultrasound energy generated by the transducers to the targeted region. The ultrasound transducers may be individually attached to the head of the patient, or in some cases, mounted within a wearable device (e.g., a cap or helmet) that is positioned on the head of the patient. In other examples, one or more ultrasound transducers that deliver ultrasound energy to the patient may be surgically attached to the patients skull (e.g., beneath the surface of the skin) to focus the ultrasound energy from the ultrasound transducers to the targeted region of the brain of the patient. Ultrasound transducers may be positioned to stereotactically deliver (e.g., precise delivery to a specific locus in a three-dimensional space) the ultrasound energy to a targeted region of the brain. One or more controller devices may control the ultrasound transducers.

The ultrasound energy may be defined by a set of ultrasound parameters selected to affect a selected region of the brain corresponding to the neurodegenerative disease, e.g., by stimulating neurons in the selected region. For example, the selected region may be a substantia nigra (SN) for a patient diagnosed with Parkinson's disease or at risk for Parkinson's disease. The ultrasound energy may stimulate the neurons within the selected region of the brain. In some examples, the targeted region of the brain to which the ultrasound energy is focused may include all, or at least a portion of, the selected region that includes the neurons at risk for degeneration. In other examples, the selected region may be separate or distinct from the targeted region. The neurons within the targeted region may be in a circuit with neurons of the selected region and thus affect the neurons of the selected region. In this manner, the selected region may be associated with the targeted region of the brain receiving the ultrasound energy.

The stimulation of neurons resulting from the delivery of ultrasound energy can provide neuroprotective effects that may prevent, reduce, or even halt, the degeneration of the neurons in the selected region of the brain that would have otherwise occurred from the neurogenerative disease. For example, the neuroprotective ultrasound energy may elicit an increase in the metabolism (e.g., increased oxygen levels) of affected brain circuits to protect selected regions of the brain from atrophy or degeneration. The ultrasound neuromodulation described herein may be considered non-invasive. In this manner, a clinician may prescribe ultrasound neuromodulation for a patient at the very earliest stages of a newly diagnosed disease (e.g., concurrent with or before medication treatment) or even responsive to the patient meeting one or more risk factors for a potential neurogenerative disease. Early application of ultrasound neuromodulation of one or more regions of the brain may prevent, reduce, halt, or even reverse the progression of one or more neurogenerative diseases and mitigate associated symptoms. Reversing the progression of a neurogenerative disease may include improving the functionality of neurons and/or neural networks within the patient and may reduce symptoms related to the neurogenerative disease.

Although the techniques primarily described in this disclosure are for reducing degeneration of neurons in one or more regions of the brain of a patient, ultrasound energy may be applied to nerves or any nervous system tissue at other locations of the body to protect such nerves or tissue from degeneration or otherwise slow the progression of a neurogenerative disease. For example, the devices, systems, and techniques described in this disclosure alternatively or additionally may be directed to other fiber tracks of the central nervous system (e.g., the spinal cord), branches therefrom, or peripheral nerves. These nerves may include sensory nerves, motor nerves, or spinal nerves.

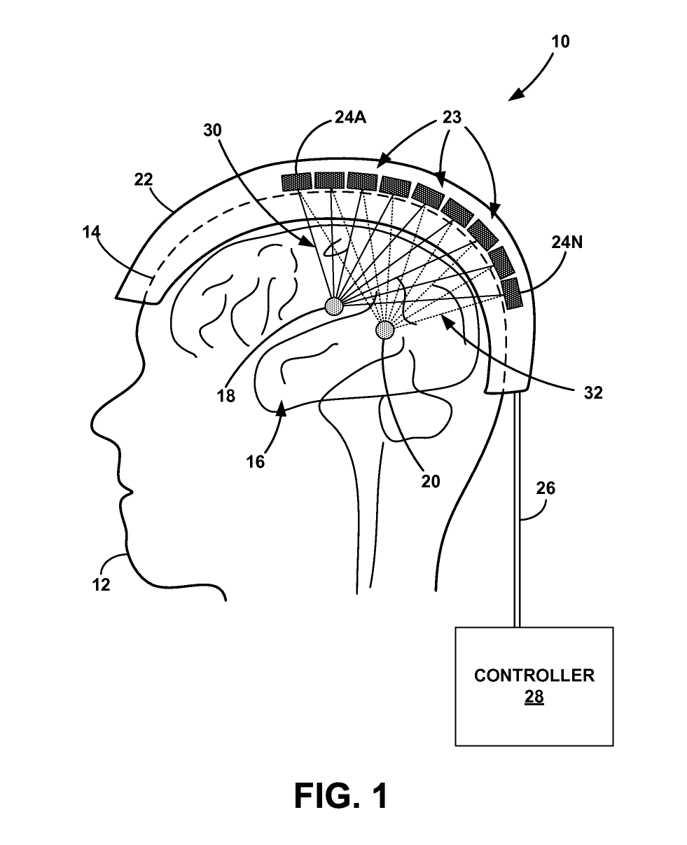

FIG. 1 is a conceptual diagram illustrating an example system 10 that delivers ultrasound energy to a targeted region of brain 16 of patient 12 to reduce neuron degeneration within a selected region of brain 16. As shown in FIG. 1, system 10 includes transducer substrate 22 in the form of a helmet or cap that may be adjustable for fitting externally along a patient's cranium 14. Substrate 22 may carry one or more ultrasound transducers 24A through 24N (collectively “ultrasound transducer array 23”). Transducers 24A through 24N may be embedded in or coated with an acoustical coupling medium to minimize signal losses between the transducer surfaces and the exterior surface of the skin of patient 12. Transducers 24A through 24N may or may not include an acoustic lens. An acoustic lens associated with a particular transducer in ultrasound transducer array 23 may be configured as an actively adjustable acoustic lens where the focal parameters may be controlled by controller device 28.

Although transducers 24A-24N are shown in a substantially straight line along cranium 14, transducers 24A-24N may be positioned at different circumferential positions around cranium 14 and/or additional ultrasound transducers may be positioned along one or more sides of cranium 14. In this manner, not all of the ultrasound transducers of array 23 may be visible in the sagittal plane cross-section of FIG. 1. In addition, the spacing between one or more transducers 24A-24N may be varied as necessary to focus ultrasound energy to appropriate targeted regions of brain 16. In some examples, the structure that carries transducers 24A-24N (e.g., helmet or cap) may allow for varying the spatial arrangement of the transducers. For instance, the special arrangement may be varied based on a particular patient's anatomy and/or based on the target region of brain 16 that is to be stimulated.

In the example of FIG. 1, transducer array 23 is coupled to controller device 28 via cable 26 for controlling ultrasound wave emission (e.g., delivery of ultrasound energy) by ultrasound transducer array 23. In other examples, controller device 28 may be attached to or embedded within substrate 22 or other portion of the helmet or cap. In other words, controller 28 may control each of ultrasound transducers 24A-24N to generate ultrasound energy according to a set of ultrasound parameters. In addition, transducer array 23 may also be coupled to a data collection module for acquiring signals from transducer array 23. The acquired signals may be signals emanated from one or more regions of brain 16 or signals in response to transmitted waves from ultrasound transducer array 23. Controller device 28 may be configured to control each of transducers 24A-24N individually. Controller device 28 may select transducers 24A-24N one at a time or in any combination for emitting ultrasound waves from ultrasound transducer array 23.

Controller device 28 may be configured to selectively control which of transducers 24A-24N are enabled for delivery of ultrasound energy (e.g., waveforms) and which transducers 24A-24N are not enabled (i.e., turned off). Controller device 28 controls the transducers of array 23 to generate waveforms corresponding to a set of ultrasound parameters. The ultrasound parameters may include, but are not limited to, identification of active ultrasound transducers, waveform shape, waveform amplitude, waveform frequency, duty cycle, the waveform phase, the number of waveforms within each burst of waveforms, and the frequency of bursts of waveforms. The waveform phase may be defined with respect to another transducer waveform, for example, a waveform generated by an adjacent transducer within array 23, a center transducer of array 23, or end transducer 24A or 24N, or another common time or clock reference.

In one example, controller device 28 may be configured to control the waveform phase of each transducer 24A-24N to select a therapy pathway for each of the individually emitting transducers 24A or 24N. For example, controller device 28 may control transducers 24A or 24N to emit ultrasound energy in the form of waveforms in a phase relationship that results in the waveforms being transmitted along pathways 30 (shown as solid lines in the example of FIG. 1) from respective transducers of array 23 to focus the emitted ultrasound energy from all of the selected emitting transducers 24A or 24N at a first targeted region 18. Controller device 28 may also adjust the phase relationship between transducers 24A or 24N to redirect the ultrasound waveforms along pathways 32 (shown as dotted lines in the example of FIG. 1) to focus the ultrasound energy at a different, second targeted region 20.

In this way, a transducer array 23 can be controlled to emit and focus ultrasound energy to reduce degeneration of neurons at one or more selected regions associated with one or more targeted regions (e.g., targeted regions 18 and 20). The volume and shape of targeted regions 18 and 20, for example, may depend in part on the number of transducers and inter-transducer waveform phase relationships selected by controller device 28.

In some examples, each of targeted regions 18 and 20 may include respective selected regions within which the ultrasound energy is intended to reduce degeneration of neurons. In other words, targeted regions 18 and 20 may include at least a portion of a respective selected region, all of a respective selected region, or even be the same as the respective selected regions. Alternatively, targeted regions 18 and 20 may be separate or distinct (e.g., non-overlapping) from the associated selected regions. Since the targeted regions 18 and 20 may affect respective selected regions via a neural circuit (e.g., neurons within a targeted region may be connected with neurons in a selected region), ultrasound energy may be focused to a targeted region (e.g., regions 18 and 20) in order to affect neurons within a different selected region of brain 16.

When multiple targeted regions are to receive ultrasound energy, controller device 28 can be configured to step through a programmed (or predetermined) set of target regions or along one or more known brain circuits to deliver the protective ultrasound neuromodulation described herein. In some examples, such predetermined sets of regions may be used to receive signals from the respective regions indicative of a brain state status (e.g., resting or elevated brain state) and/or to identify the desired target or selected regions. Target regions may be selected one at a time in a sequential manner or selected two or more at time for simultaneous neuromodulation at more than one target region. A menu, or set, of target regions used by controller device 28 to select one or more transducers 24A-24N may include regions selected one at a time or in combination, in any desired order. In addition, the target regions may be selected based on one or more selected regions for which the neurons may be at risk of degeneration. In other words, the target regions may be selected in order to affect one or more selected regions.

In some examples, controller device 28 may control array 23 to operate in a receiving mode for measuring reflections of ultrasound waves for use as feedback in focusing ultrasound energy on a target region and/or for measuring a functional response (e.g., a brain state) to neuromodulation. Controller device 28 may control array 23 to emit ultrasound waveforms for neuroprotective purposes and, in some examples, measure reflections of the ultrasound waveforms using the same transducer(s). Alternatively, controller device 28 controls array 23 to emit neuroprotective ultrasound waveforms and imaging ultrasound waveforms using two different sets of waveform emission control parameters. Controller device 28 may control array 23 to alternate between neuroprotective and imaging waveform emissions, in which different ultrasound parameter sets are used to define the neuroprotective waveforms and the imaging waveform.

Emitted waveforms may have a frequency ranging between approximately 0.1 MHz and 20 MHz. Neuroprotective ultrasound waveforms may have a relatively low frequency in a range of approximately 0.1 MHz to 5 MHz. In some examples, neuroprotective ultrasound waveforms may be between 0.1 MHz and 1 MHz. In other examples, the frequency range may be between approximately 0.1 MHz to 0.3 MHz to target neurons within the skull. Imaging ultrasound waveforms may have a relatively higher frequency in the range of approximately 2 MHz to 20 MHz. In some examples, imaging ultrasound waveforms may be between approximately 2 MHz and 10 MHz. The frequency ranges for neuroprotective waveforms and imaging waveforms may overlap in some examples, and any reflections received by one or more transducers of array 23 may be measured by system 10 (e.g., controller device 28 or another imaging module) for generating image data of one or more regions within brain 16.

Higher frequency ultrasound waveforms may be used to generate more detailed imaging data than frequencies used to deliver neuroprotective ultrasound waveforms. In this case, controller device 28 may control array 23 to emit distinct neuroprotective waveforms and imaging ultrasound waveforms, which may be delivered simultaneously or in an alternating manner. The imaging waveforms may be delivered by selecting the same or different transducers within array 23 as the transducers selected for delivering neuroprotective waveforms. The imaging waveforms may be less focused than neuroprotective waveforms to obtain a larger view of an anatomical region or focused on a different region of brain 16 than a targeted region receiving neuroprotective energy in order to monitor a functional response at the different region. Neuroprotective energy may be the energy provided by ultrasound waves that protects neurons, such as dopaminergic neurons, from dopamine depleting neurotoxins.

Controller device 28, or a different device, may measure reflections of the imaging waveforms and may generate image data. In some examples, the reflections of the neuroprotective waveforms may be measured in addition to the reflections of the imaging waveforms for collecting data relating to a target region, relating to the pathways 30 and 32, and/or relating to a functional response (e.g., a brain state that indicates a level of neuron activity) to the neuroprotective waveforms at the selected region intended to receive the neuroprotective effects or a different region (e.g., a target region to which the neuroprotective waveforms are focused).

Although controller device 28 may generate image data from reflected ultrasound waves, data indicative of a brain state of a region in brain 16 may be received using other techniques. For example, system 10 may include one or more electrodes configured to receive electrical signals indicative of neuron activity (e.g., an electroencephalogram (EEG). The electrical signals may be received from independent brain activity and/or in response to ultrasound waves or electrical signals delivered to the region of brain 16. For example, signals indicating an elevated brain state at the selected region may indicate that delivered ultrasound waves are affecting the desired selected region. In this manner, system 10 may utilize alternative sensors and/or delivery modalities from ultrasound transducers in some examples.

A targeted region to which neuroprotective ultrasound waves are focused may be different than a region that is imaged. The neuroprotective ultrasound energy may be delivered at the target region while functional imaging is performed at a monitoring region, i.e., a selected region of interest, to measure a change in response to the delivered ultrasound energy. For example, a response to neuroprotective ultrasound energy may include a change in tissue density due to a blood flow change at the selected region or at a different monitoring region. Accordingly, controller device 18 may use ultrasound parameters that cause array 23 to emit imaging waveforms for imaging and include transducer selection, waveform phase, or other parameters that influence focusing of the imaging waveforms. The size of the monitoring region may be the same as or different (e.g., larger or smaller) than the size of the targeted region, and the monitoring region may or may not include the targeted region. The focusing resolution(s) and target(s) for targeted regions for neuroprotective waves and monitoring regions can be defined separately in a menu or set of regions within brain 16 to be tested. Controller device 28 may control array 23 to achieve neuroprotective wave delivery at targeted region(s) to affect selected regions and monitor for a response to therapy at imaging regions selected to correspond to an expected response to the neuroprotective energy delivered to the targeted regions. In some examples, the imaging regions may be the selected regions of brain 16 that include neurons intended to be protected by the neuroprotective ultrasound energy.

Array 23 may, in some examples, include dedicated ultrasound transducers that deliver neuroprotective ultrasound waves and dedicated transducers (e.g., imaging transducers) that receive reflected ultrasound waves. Controller device 28 may be configured to control the imaging transducers to operate simultaneously or in alternating fashion with delivery transducers that deliver the neuroprotective ultrasound waves. As such, array 23 may include two or more sub-arrays, which may include one or more dedicated therapy delivery array(s) and one or more dedicated imaging array(s). The functionality of the transducers 24A-24N, however, may be completely programmable and flexible as controlled by controller device 28.

Controller device 28 may be programmed or otherwise receive instructions by another programming device. Controller device 28 may communicate with the programming device via a wired or wireless communication protocol. In addition, or alternatively, controller device 28 may also include a user interface that receives input from a user (e.g., a clinician, technician, or patient 12) and/or outputs data related to the obtained information or currently used ultrasound parameter sets or associated programs. In some examples, the user interface includes, for example, a keypad and a display, which may, for example, be a liquid crystal display (LCD) or light emitting diode (LED) display. The keypad may take the form of an alphanumeric keypad or a reduced set of keys associated with particular functions. Controlling device 28 can additionally or alternatively include a peripheral pointing device, such as a mouse or stylus, via which a user may interact with the user interface. In some examples, a display of controller device 28 may include a touch screen display, and a user may interact with controller device 28 via the display. It should be noted that the user may also interact with controller device 28 remotely via a networked computing device. In other examples, controller device 28 may interface with a separate computing device (e.g., a mobile computing device or workstation) that interfaces with the clinician.

As described herein, system 10 may be configured to reduce neuron degeneration within brain 16 of patient 12. For example, system 10 may be configured to deliver, via one or more ultrasound transducers 24A-24N, ultrasound energy focused to a targeted region (e.g., targeted regions 18 and 20) of brain 16 of patient 12. System 10 may deliver the ultrasound energy according to a set of ultrasound parameters selected to cause one or more of ultrasound transducers 24A-24N to generate ultrasound energy that reduces neuron degeneration within a selected region (not shown) of brain 16. The selected region may be associated with one or both of targeted regions 18 and 20. In some examples, controller device 28 may include one or more processors that control an ultrasound module to provide signals that modulate the operation of ultrasound transducers 24A-24N.

Controller device 28 may also select one or more ultrasound parameters values for the set of ultrasound parameters that at least partially define delivery of ultrasound energy focused to the targeted region(s) of brain 16. For example, controller device 28 may select the ultrasound parameter values based on one or more selected regions to receive neuroprotective effects of the ultrasound energy and/or one or more targeted regions to which the ultrasound energy is to be focused. In some examples, controller device 28 may automatically select the ultrasound parameter values according to regions of the brain identified by a clinician or other user. In other examples, controller device 28 may select one or more ultrasound parameter values in response to signals received from targeted regions and/or selected regions within brain 16. Controller device 28 may titrate ultrasound energy to identify appropriate parameter values that may achieve neuroprotective effects for a certain selected region and select those appropriate parameter values, as described in more detail below. Alternatively, controller device 28 may select the ultrasound parameter values based on a program or other instructions received by a clinician or patient.

The set of ultrasound parameters may specify one or more aspects of delivery of the neuroprotective ultrasound energy. For example, the ultrasound parameters may define the one or more ultrasound transducers of array 23 that are to deliver ultrasound waves, waveform shape, waveform amplitude, waveform frequency, duty cycle, waveform phase, and start and stop times of ultrasound energy. In this manner, ultrasound parameters may define the ultrasound waves delivered to patient 12 and when each of the ultrasound transducers of array 23 is activated to generate such ultrasound waves.

The process of delivering neuroprotective ultrasound energy may also include positioning the one or more ultrasound transducers of array 23 on an external surface of a head (e.g., cranium 14) of patient 12 to focus the ultrasound energy from the ultrasound transducers to targeted regions 18 and 20. In some examples, positioning the one or more ultrasound transducers of array 23 may include positioning transducer substrate 22 on cranium 14 of patient 12. A clinician or patient 12 may align one or more locations of substrate 22 with landmarks on the head of patient 12, such as the ears, temples, or other locations. In other examples, substrate 22 may be pre-conformed to cranium 14 such that substrate 22 only fits correctly in one position. In other examples, a clinician or patient 12 may need to place ultrasound transducers 24-24N, either individually or in groups, at the appropriate locations on cranium 14. Positioning of ultrasound transducers 24A-24N may be performed to focus the ultrasound transducers to a certain targeted region within brain 16. Positioning ultrasound transducers 24A-24N may be an iterative process in which ultrasound transducers emit imaging waves, detect reflected waves resulting from the emitted imaging waves, and determine that the current ultrasound transducer location is satisfactory or unsatisfactory to focus ultrasound energy to the targeted region. In this manner, the one or more ultrasound transducers 24A-24N may form an array of ultrasound transducers positionable at respective locations on an external surface (e.g., cranium 14) of a head of patient 12.

As discussed herein, targeted regions and selected regions of brain 16 may include the same, at least partially overlapping, portions of brain 16 or separate or distinct portions of brain 16. Targeted regions are those regions of brain 16 to which ultrasound energy is focused. Selected regions of brain 16 are those regions that contain neurons intended to be protected by the neuroprotective ultrasound energy. In other words, a selected region typically includes neurons that would otherwise degenerate due to a neurodegenerative disorder without the delivery neuroprotective ultrasound energy. A targeted region may be the same as a selected region in some examples. In other examples, the targeted region of the brain may include at least a portion of the selected region of the brain such that the targeted region overlaps with the selected region and/or the targeted region and the selected region share at least one common neuron.

In another example, a targeted region is different than a selection region of brain 16, but the targeted region of brain 16 may include neurons that affect different neurons within the selected region of brain 16. Neurons within brain 16 may be connected to form a brain circuit such that neuron activity in one region of brain 16 affects the activity of another neuron in another different region of brain 16. In this manner, system 10 may focus ultrasound energy to a targeted region in order to affect, or modulate, neuron activity in a selected region at a different location within brain 16. Such connections may allow system 10 to provide neuroprotective benefits to neurons within the selected region of brain 16 and limit the exposure of neurons of the selected region to ultrasound waves. In addition, the targeted region of brain 16 may be superficial to an associated selected region of brain 16. Since system 10 may be able to focus ultrasound waves of lower energy to more superficial neurons (e.g., neurons closer to the surface of scalp 14) than deeper neurons, system 10 may focus ultrasound waves with less energy to the more superficial targeted regions of brain 16 in order to affect one or more selected regions deeper within brain 16. In other words, higher energy ultrasound waves (e.g., lower ultrasound frequencies) may be required to focus ultrasound waves to deeper regions in brain 16 than more superficial regions of brain 16.

In some examples, controller device 28 may select the one or more ultrasound parameters for the set of ultrasound parameters at least partially defining neuroprotective ultrasound energy via an iterative process. This iterative process may be referred to as titrating ultrasound energy. For example, a processor of controller device 28 may be configured to receive a first signal from the selected region of the brain and associate the first signal with a resting state of the selected region of the brain. The first signal may be indicative of reflected imaging ultrasound waves that have reflected off of the selected region of brain 16. In other examples, the first signal may be indicative of an electroencephalogram (EEG) representative of electrical activity of the neurons within the selected region. In any case, the first signal may be indicative of the resting state of the selected region that occurs without delivery of ultrasound energy. The resting state may be the baseline brain state used to identify an elevated brain state as described below.

Controller device 28 may select a first set of ultrasound parameter values as an initial ultrasound parameter set and deliver ultrasound energy to a targeted region (e.g., targeted region 18 or 20) of brain 16 according to the initial ultrasound parameter set. The initial ultrasound parameter set may be a predetermined parameter set that may or may not be specific to the location of the selected region in brain 16. The processor of controller device 28 may then iteratively adjust values of one or more of the ultrasound parameters (e.g., ultrasound wave amplitude, waveform shape, and/or duty cycle) until the processor receives a second signal from the selected region of the brain indicative of an elevated state of the selected region of the brain. The elevated state may indicate that the ultrasound energy is stimulating the neurons of the selected region at too high a level, and the neuron activity associated with neuroprotection may be just below the detected elevated state. A brain state just below the elevated state may be below a perception threshold at which the patient perceives the ultrasound therapy and above an activation threshold at which neurons are activated. However, the activity of the neurons may be insufficient to elevate the brain state of that particular region of the brain. Neuroprotective ultrasound energy may thus protect neurons from dopamine depleting neurotoxins through increased activity, but neuroprotective ultrasound energy may not be configured to produce an immediate therapy or perceivable therapeutic effect reducing symptom frequency or severity. The neuroprotective ultrasound energy is instead configured to protect remaining neurons from further degeneration. In some examples, the neuroprotective ultrasound energy may elicit an increase in the metabolism of affected brain circuits to protect selected regions of the brain from atrophy or degeneration.

In response to detecting the elevated state of brain 16, the processor of controller device 28 may select previous values of the one or more ultrasound parameters as a final ultrasound parameter set. In other words, controller device 28 may use the ultrasound parameters from the last iteration of ultrasound energy that did not result in the elevated state of brain 16 as the final ultrasound parameter set. Controller device 28 may then deliver the neuroprotective ultrasound energy according to the final ultrasound parameter set. In some examples, the neuroprotective ultrasound energy is configured to be below a perception threshold at which patient 12 perceives delivery of the ultrasound energy and also below an activation threshold at which neurons within the selected region of brain 16 are activated. In other examples, the neuroprotective ultrasound energy may be configured to be below either the perception threshold or the activation threshold. In this manner, controller device 28 may request patient feedback during delivery of neuroprotective ultrasound energy to determine if patient 12 can perceive any effects, and controller device 28 may adjust one or more ultrasound parameter values to reduce or eliminate and perceived effects.

As described herein, neuroprotective ultrasound energy may be delivered to reduce nerve or neuron degeneration associated with one or more diseases or disorders. For example, the set of ultrasound parameters may be selected to generate ultrasound energy configured to reduce nerve and/or neuron degeneration associated with Alzheimer's disease, Parkinson's disease, tremor, or dystonia, dementia, or chronic pain. Each of these diseases or disorders may be associated with typical regions within the brain at which neurons degenerate over time. These typical regions may be identified as the selected regions for neuroprotective ultrasound energy. For example, Parkinson's disease may be associated with the Substantia nigra and/or subthalamus nucleus. In some examples, targeted regions to which the ultrasound energy is focused may be determined in order to affect the selected regions associated with the disease or disorder of the patient.

Neuroprotective ultrasound energy may be delivered to a patient at any time to reduce the degeneration of brain nuclei, fiber tracks and/or neurons. However, early delivery of neuroprotective ultrasound energy may increase the amount of time a patient retains motor function and/or cognitive function. In this manner, a clinician may prescribe neuroprotective ultrasound energy in response to the first diagnosis of a neurodegenerative disease or disorder. The clinician may prescribe neuroprotective ultrasound energy with system 10 concurrently with medication and/or before medication is even prescribed. Since system 10 may operate non-invasively, there may be little to no risk to early delivery of the neuroprotective ultrasound energy.

In other examples, a clinician may prescribe neuroprotective ultrasound energy delivery for patients at risk of contracting a neurodegenerative disease and before any disease symptoms occur or the disease can otherwise be diagnoses. For example, the clinician may monitor one or more risk parameters for one or more respective neurodegenerative disease. If one or more risk parameter values exceed the respective threshold for a neurodegenerative disease, the clinician may prescribe neuroprotective ultrasound energy delivery. In some examples, a single risk parameter value exceeding the threshold may be sufficient for neuroprotection. In other examples, a predetermined number of risk parameter values may need to exceed their respective thresholds before a clinician may prescribe neuroprotective ultrasound energy delivery. Example risk factors may include genetic history, environmental conditions predisposing the patient to a neurodegenerative disorder, measured decrease in the volume of a selected region of the brain, age, and/or injuries known to lead to one or more degenerative diseases.

System 10 may be configured to deliver neuroprotective ultrasound energy to patient 12 at only certain times of the day. For example, a clinician may prescribe neuroprotective ultrasound energy to be delivered during a sleep period of patient 12 to reduce any impact to daily routine and/or reduce any perceived side attributed to the ultrasound energy. In other examples, patient 12 may need to receive the neuroprotective ultrasound energy at multiple times during the day. The amount of time patient 12 needs to receive neuroprotective ultrasound energy may depend upon the detected progression of the disease or patient feedback. If patient 12 is experiencing a greater number of symptoms, patient 12 may request an increase delivery time for the neuroprotective ultrasound energy each day, for example.

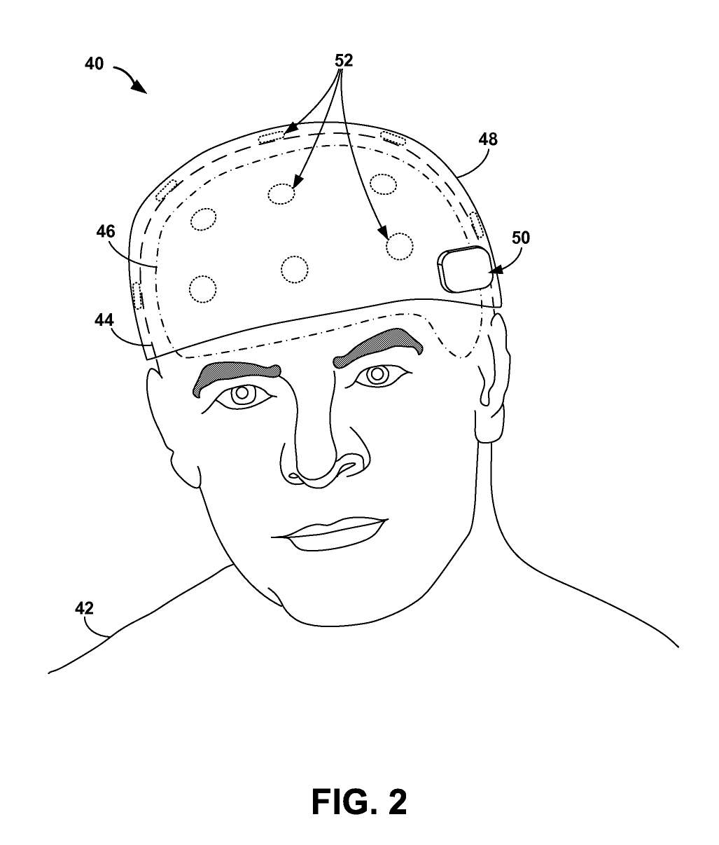

FIG. 2 is a conceptual diagram illustrating an example wearable device 40 that includes an array of ultrasound transducers 52 that deliver ultrasound energy to a targeted region of brain 46. Wearable device 40 may be similar to system 10 of FIG. 1 and patient 42 may be similar to patient 12. As shown in FIG. 2, wearable device 40 may include substrate 48, an array of ultrasound transducers 52, and controller device 50. Substrate 48, ultrasound transducers 52, and controller device 50 may be similar to substrate 22, ultrasound transducers 24A-24N, and controller device 28, respectively, of FIG. 1. Patient 42 may wear wearable device 40 during a sleep session, when awake at home, or at any other location.

Wearable device 40 may represent a helmet, hat, cap, or other article that is configured to be worn over cranium 44 of patient 42. Substrate 48 may be constructed of one or more types of polymers, fabrics, or other materials. In some examples, the different materials of substrate 48 may be layered. For example, an interior layer may be constructed of a flexible polymer configured to conform to the skin surface of cranium 44. An exterior layer may be more durable and more rigid than the interior layer to allow a suitable fixation structure for each of transducers 52 and/or controller device 50. In some examples, breathable fabric may be disposed between the skin of patient 42 and a flexible polymer layer to accommodate patient 42 wearing wearable device 40 for several hours or even longer. Wearable device 40 may be constructed specifically for patient 42 or to be worn by many different patients.

Each of ultrasound transducers 52 may be mounted to or embedded at least partially within substrate 48. The position of ultrasound transducers 52 within substrate 48 may be selected such that each of ultrasound transducers 52 contacts the skin of cranium 44 when wearable device 40 is placed on cranium 44. In this manner, an energetic surface of the ultrasound transducers 52 may be disposed flush with an interior surface of substrate 48 or protruding from the interior surface of substrate 48. In general, wearable device 40 may include between two and 1000 ultrasound transducers 52. In one example, wearable device 40 may include between 5 and 20 ultrasound transducers 52. Example numbers of ultrasound transducers 52 may be 5, 10, or 20 transducers. In one example, the ultrasound transducers may be replaced with microscopic Capacitive Machined Ultrasound Transducers (CMUTs). The number of CMUTs may be greater than one million in some examples. Groups of CMUTs may be wired in parallel to create functional transducer blocks in which each CMUT of the group behaves identically. The spatial arrangement of the transducers or transducer blocks on substrate 48 may be patient-specific (based on the size and/or shape of a particular patient's cranium 44 or on an intended target) or may instead be more generic.

Each of ultrasound transducers 52 may be identical. Alternatively, some of ultrasound transducers 52 may have a different size, shape, or even be tuned for different ultrasound frequencies than other ones of ultrasound transducers 52. In this manner, each ultrasound transducer within substrate 48 may be configured for the specific location at which the transducer is disposed within the substrate. The types of ultrasound transducers may be selected to focus to a specific region of brain 46 (e.g., depth of the region and/or volume of the region) and/or based on the thickness of the skull through which the ultrasound waves must penetrate to reach the targeted region. The type of ultrasound transducers 52 may be tailored to patient 42 and/or the targeted regions to which ultrasound energy will be focused.

Controller device 50 may also be partially or fully embedded within substrate 48 or otherwise attached to substrate 48. Controller device 50 may be electrically connected to each of ultrasound transducers 52 and include an ultrasound module configured to energize and/or receive signals from the ultrasound transducers. Controller device 50 may also include a processor configured to control the ultrasound module and perform other tasks related to delivery of neuroprotective ultrasound energy. Controller device 50 may include a power source (e.g., rechargeable and/or replaceable battery) and a user interface that allows a clinician or patient to adjust at least some operations of controller device 50.

A telemetry module within controller device 50 may also allow a programming device to transmit instructions to and/or receive data from controller device 50. The programming device may be a dedicated handheld computing device, a mobile device (e.g., a mobile phone or tablet computing device), a notebook computer, a workstation, or any other computing device configured to communicate with controller device 50. In some examples, controller device 50 and/or the programming device may communicate with a remote server or other remove computing device via a network. In this manner, a clinician may remotely interact with the operation of controller device 50.

Although external devices with ultrasound transducers may be used as described herein, ultrasound transducers may be implanted in some examples. For example, some or all of the ultrasound transducers may be implanted beneath the skin of the scalp and external of the cranium during a minimally invasive procedure. A controller device 50 and other electronics may be included within an implantable medical device coupled to the ultrasound transducers such that no percutaneous ports are necessary for operation. Alternatively, one or more percutaneous leads may couple the implanted ultrasound transducers with an external control circuitry. In other examples, the only implanted element may include a positioning device that positions one or more externally placed ultrasound transducers to the head of the patient. For example, one or more small magnets may be implanted beneath the scalp of the patient. A corresponding magnet associated with an external one or more ultrasound transducers (e.g., the corresponding magnet constructed into an array of ultrasound transducers) may then couple to the implanted one or more magnets to maintain proper alignment of the external ultrasound transducers to deliver ultrasound energy to the targeted region of the brain of the patient.

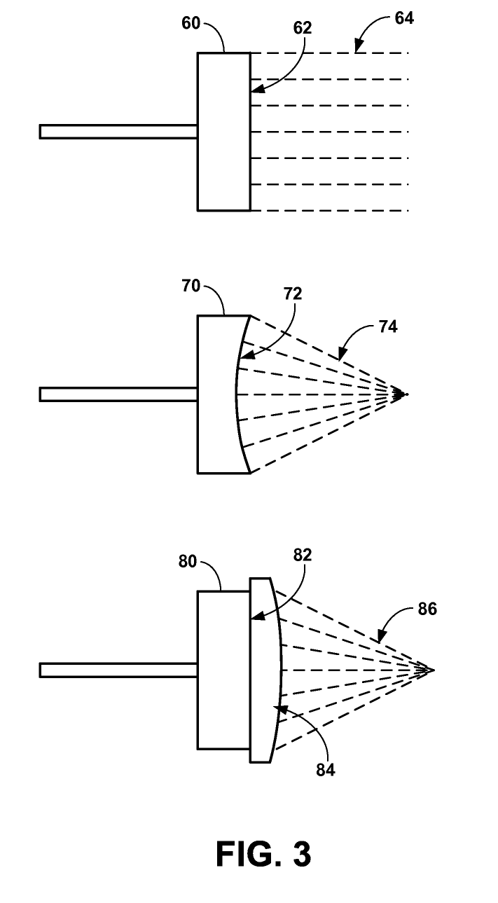

FIG. 3 is a conceptual diagram illustrating example ultrasound transducers 60, 70, and 80 that can be used to focus ultrasound energy to a targeted region of a brain. Any of ultrasound transducers 60, 70, or 80 may be implemented in system 10 of FIG. 1 or wearable device 40 of FIG. 2. Transducer 60 is shown having a flat acoustical surface 62 such that emitted waveforms 64 are unfocused. In other words, emitted waveforms 64 may be emitted generally perpendicular from flat acoustical surface 62. Transducer 70 includes a concave acoustical surface 72 emitting focused ultrasound waves 74 to a particular location at a specified distance from concave acoustical surface 72. Transducer 80, having a flat acoustical surface 82, includes an acoustical lens 84 (e.g., a convex lens) for focusing ultrasound waves 86 to a particular location at a specified distance from flat acoustical surface 82.

Ultrasound transducers of the types illustrated by transducers 60, 70, and 80, or other types of ultrasound transducers, may be used solely or in any combination in a transducer array for delivering neuroprotective ultrasound energy and/or imaging ultrasound energy. In some examples, certain types of transducers may be used to deliver neuroprotective ultrasound waves and other types of transducers may be used to deliver and/or receive ultrasound waves for imaging or functional diagnostic purposes. The waveforms emitted by a combination of transducers may be focused at a target region using ultrasound parameters such as phase relationships. As used herein, a transducer “array” refers to any n×n array, wherein n may be 1 or greater than 1, the array including multiple transducers or a 1×1 array with a singular transducer. A transducer array is not limited to a linearly arranged array, or an array arranged in rows and columns, but may include, for example, a circular array, a random array or any other arrangement of transducers along a substrate.

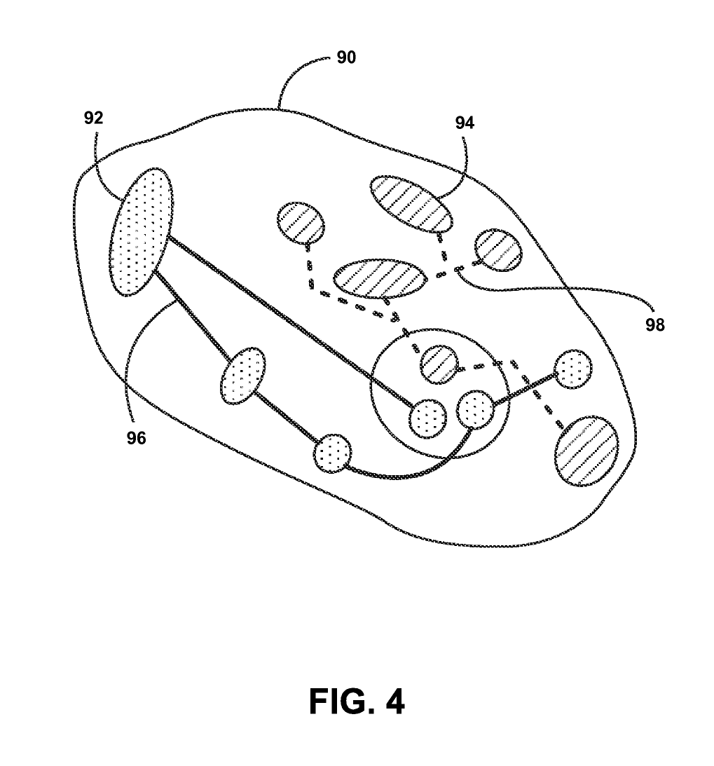

FIG. 4 is a schematic diagram of example regions and brain circuits within brain 500 of a patient. A first brain circuit 96 shown by a solid line in FIG. 4 links multiple brain structures 92 (shown as dotted regions). A second brain circuit 98 links brain structures 94 (shown as stripped regions). An ultrasound transducer selection protocol may be defined to select ultrasound transducers that will enable system 10, for example, to focus neuroprotective ultrasound waveforms on one or more structures 92 and 94 of a respective brain circuit 96 and 98 and along known neural pathways of these circuits. Neuroprotective ultrasound energy may be focused to a target region along a brain circuit in order to affect neurons within a different selected region within the same brain circuit. In other words, brain circuits such as example brain circuits 96 and 98 may allow neuroprotective ultrasound energy to be focused to a targeted region of the brain to affect neurons within a different selected region of the brain. For example, neuroprotective ultrasound waves focused on a superficial region within brain circuit 96 may affect a selected region deep within brain 90 and within the same brain circuit 96. Alternatively, or additionally, neuroprotective ultrasound waves may be focused to one or more target regions within one or more brain circuits while acquiring functional imaging data to provide detailed diagnostic data of other selected regions within the respective brain circuits.

While the examples of FIG. 4 primarily focus on the brain as the target, it will be understood that some or all of the techniques described herein may be applied to any other area of the anatomy that may be the target of an electrical stimulation therapy, an ultrasound therapy, a drug delivery therapy, or any combination thereof. Such therapies or procedures may be delivered acutely or chronically. A chronic therapy is a therapy used for more than one day, for example, and may be delivered using external and/or implantable therapy delivery devices.

Targets for acute or chronic neuroprotective ultrasound stimulation delivery may include but are not limited to, the following: spinal nerves for back pain, intercostal nerves for mastectomy pain, sciatic nerve for muscular constriction, supra/suborbital/infraorbital nerves and trigeminal nerve for facial pain, cranial nerves for cervical pain, median nerve for carpal tunnel, cluneal/iliohypogastric/lateral femoral nerves for pain associated with iliac bone crest harvest, ilioinguinal and iliohypogastric nerves for herniorrhaphy pain, vagus nerve for vagus nerve stimulation for treating epilepsy, hypertension and depression and occipital nerves for chronic migraine. Urinary frequency and urgency, fecal incontinence, chronic pelvic pain, painful bladder syndrome, interstitial cystitis, chronic prostatitis, and sexual dysfunction may be treated with any combination of delivery of ultrasound stimulation to sacral nerves, pudendal nerve and its branches, tibial nerve and its branches, dorsal nerve of clitoris for females, and dorsal nerve of penis for males. In some examples, ultrasonic stimulation may be delivered to excite nerves for exercising muscles following spinal cord injury. Neuroprotective ultrasound energy may be combined with electrical, pharmaceutical or other neuromodulation techniques, and may target a different modulation site. For example, ultrasound modulation of a peripheral nerve site may be combined with electrical stimulation of a central nervous system site or vice versa.

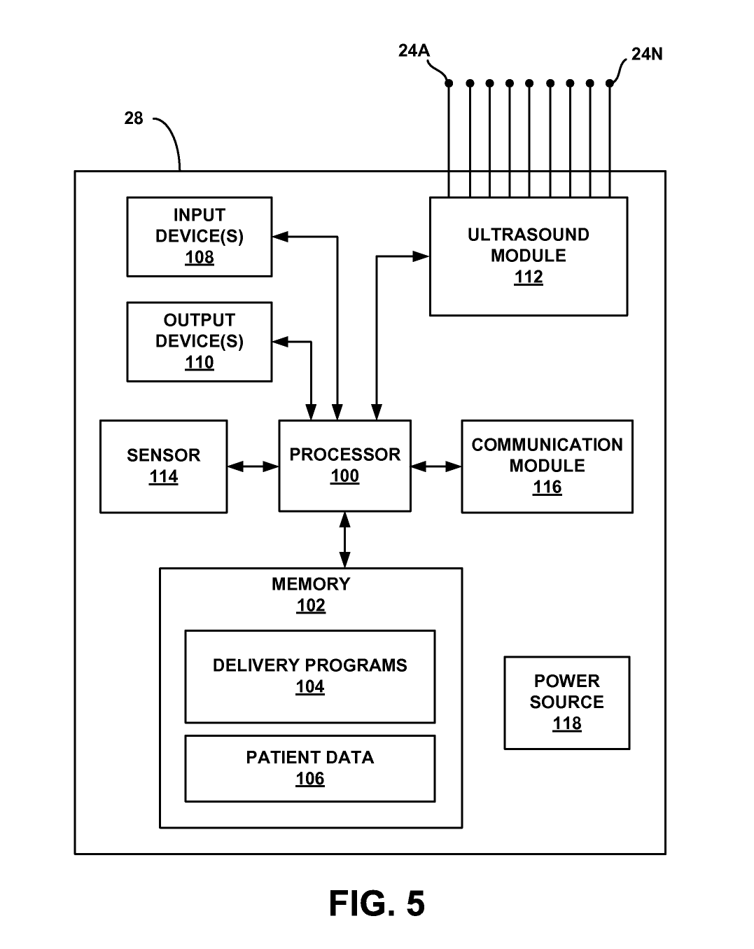

FIG. 5 is a block diagram illustrating an example configuration of controller devices 28 or 50 FIGS. 1 and 2, respectively. In the example of FIG. 5, controller device 28 includes processor 100, memory 102, ultrasound module 112, sensor 114, input devices 108, output devices 110, communication module 116, and power source 118. In other examples, controller device 28 may include more or fewer components. For example, controller device 28 may include electrodes and sensing circuitry for receiving electrical signals from the brain and generating an EEG from the received signals. In other examples, sensor 114 and/or one or more of input devices 108 or output devices 110 may not be included.

In general, controller device 28 may comprise any suitable arrangement of hardware, alone or in combination with software and/or firmware, to perform the techniques attributed to controller device 28 and processor 100 and ultrasound module 112 of controller device 28. In various examples, controller device 28 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. Controller device 28 also, in various examples, may include a memory 102, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Moreover, although processor 100 and ultrasound module 112 are described as separate modules, in some examples, processor 100 and ultrasound module 112 (or more devices of controller device 28) are functionally integrated. In some examples, processor 100 and/or a separate controller for ultrasound module 112 correspond to individual hardware units, such as ASICs, DSPs, FPGAs, or other hardware units.

Memory 102 stores information such as instructions and generated data. For example, memory 102 may include delivery programs 104 and patient data 106. Delivery programs 104 may include instructions (e.g., one or more programs) that define the delivery of neuroprotective ultrasound energy. Each program may include respective a set of ultrasound parameters that defines which ultrasound transducers of an array are active, waveform shape, waveform amplitude, waveform frequency, duty cycle, the waveform phase, the number of waveforms within each burst of waveforms, and the frequency of bursts of waveforms. Each delivery program 104 may also define when the neuroprotective ultrasound energy should be delivered (e.g., certain times of day, duration of delivery, days of the week, etc.). In some cases, the time at which ultrasound energy should be delivered is coordinated with, or otherwise based on, the time of delivery of another therapy, such as the delivery of a medication. Memory 102 may also include programs that define determination of appropriate ultrasound parameters, ultrasound imaging processes, and/or the determination of brain states of selected regions of the brain.

Patient data 106 may include data generated during the operation of controller device 28. For example, patent data 106 may include times and durations at which neuroprotective ultrasound energy was delivered, detected brain states, targeted regions and selected regions, imaging information from reflected ultrasound waves, EEG information, or any other data. Processor 100 may, in some examples, output patient data 106 for presentation to the user via output devices 110 and/or via for communication by communication module 116 to a different computing device. Patient data 106 may also include any detected errors that occurred during the delivery of neuroprotective ultrasound energy.

Ultrasound module 112 is configured to energize any of ultrasound transducers 24A-24N according to the ultrasound parameters stored in delivery programs 104. For example, processor 100 may control ultrasound module 112 to apply electrical signals to one or more of ultrasound transducers 24A-24N to generate neuroprotective ultrasound waves. Alternatively, ultrasound module 112 may independently control the ultrasound transducers using the stored ultrasound parameters. In some examples, ultrasound module 112 may also receive signals generated by one or more of ultrasound transducers 24A-24N in response to detecting reflected waves. Ultrasound module 112 may then generate data indicative of the received signals for purposes such as generating imaging information regarding targeted regions and/or selected regions within the brain.

Sensor 114 may be a temperature sensor that detects the temperature of tissue adjacent to controller device 28 and/or the temperature of the skin adjacent to one or more of ultrasound transducers 24A-24N. Processor 100 may monitor a signal from sensor 114 and terminate the delivery of ultrasound energy if the signal indicates that the temperature exceeds a predetermined threshold. Processor 100 may also redeliver ultrasound energy in response to determining that the temperature falls back below the threshold. In addition, or alternatively, sensor 114 may include one or more accelerometers that generate a signal indicative of patient movement. Processor 100 may monitor the motion of the patient to determine when the patient falls asleep and may begin delivery of neuroprotective ultrasound energy in response to determining that the patient is asleep. In this manner, processor 100 may limit the ultrasound energy from potentially disrupting sleep. In other examples, controller device 28 may include an electroencephalogram (EEG) module configured to receive electrical signals from electrodes placed on the cranium of the patient and generate EEG signals. Processor 100 may determine brain states (e.g., resting states or elevated states) according to the generated EEG signals. In one example, processor 100 may utilize the EEG signals instead of, or in addition to, the accelerometer signal(s) to determine when the patient has fallen asleep so that ultrasound energy may be delivered when the patient is asleep.

Controller device 28 may be configured to receive inputs from a user. Input devices 108 may include one or more buttons, keypads, touch-sensitive screen, pointing device, or any other input device. Output devices 110 may include one or more lights, a speaker, and a display, such as a liquid crystal (LCD), light-emitting diode (LED), or cathode ray tube (CRT). In some examples the display may be a touch screen. Output devices 110 may thus be configured to output information (e.g., the status of neuroprotective ultrasound energy delivery and/or patient data 106) to a user. Processor 100 may be configured to control input devices 108 and output devices 110. For example, processor 100 may control output device 104 to present an indication of whether or not ultrasound energy is being delivered. Processor 100 may also control output devices 110 to present any information associated with the delivery of ultrasound energy or the operational status of controller device 28. In some examples, processor 100 may control output devices 110 to indicate any malfunction of a transducer or controller device 28. In some examples, the combination of input devices 108 and output devices 110 may be referred to as a user interface for controller device 28.

Communication module 116 may be configured to receive data from another computing device and/or transmit data to another computing device. Communication module 116 may be configured to communicate via wired or wireless communication protocols for direct communication or via a network. Examples of wireless communication techniques that may be employed to facilitate communication between controller device 28 and another computing device include RF communication according to the 802.11 or Bluetooth specification sets, infrared communication, e.g., according to the IrDA standard, or other standard or proprietary telemetry protocols.

Power source 118 delivers operating power to the components of controller device 28. Power source 118 may include a battery and a power generation circuit to produce the operating power. In some examples, the battery may be rechargeable to allow extended operation. In other examples, power source 118 may be configured to accept replaceable batteries or receive power from an alternating current (AC) outlet.

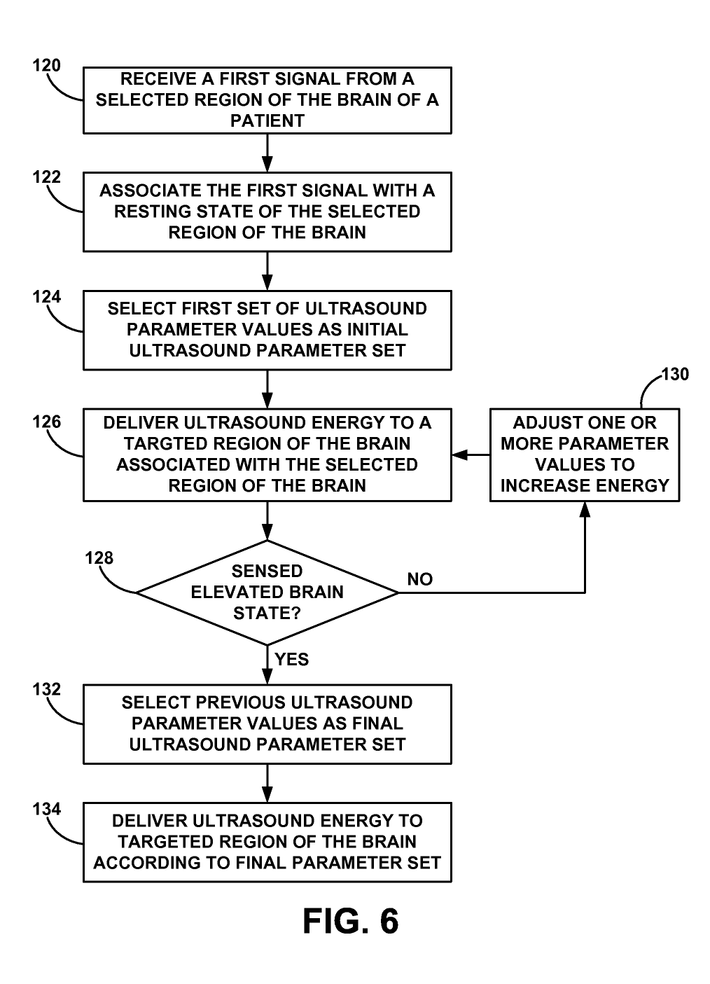

FIG. 6 is a flow diagram that illustrates an example process for determining a set of ultrasound parameters that at least partially define ultrasound energy deliverable to a targeted region of a brain of a patient. As described in FIG. 6, processor 100 of controlling device 28 may be used to determine ultrasound parameters for use in delivering neuroprotective ultrasound energy to a targeted region of brain 16. However, in other examples, other devices or systems, such as wearable device 40, may be used to perform such a process. In some examples, controlling device 28 may perform the process of FIG. 6 in response to a single input received from a patient requesting the determination of an appropriate set of ultrasound parameter (e.g., controlling device 28 may autonomously determine the set of ultrasound parameters).

Processor 100 may select a region (e.g., a selected region) that includes neurons that may be subject to degeneration from a neurodegenerative disease. For example, the substantia nigra (SN) may be the selected region for a patient diagnosed with Parkinson's disease. The selected region may be determined by processor 100 or a user (e.g., a clinician or user) based on a neurodegenerative disease for which the patient has been diagnosed or is otherwise at risk. Processor 100 may receive a first signal from the selected region of brain 16 of patient 12 (120). The first signal may be received without delivery of ultrasound energy affecting the neurons in the selected region. The first signal may be representative of an EEG of the selected region or any other diagnostic modality. Processor 100 may then associate the first signal with a resting state of the selected region of brain 16 (122).

Processor 100 may then select a first set of ultrasound parameter values as an initial ultrasound parameter set (124). The initial ultrasound parameter set may be generic for any selected regions or targeted regions or specifically tailored for a respective selected region or targeted region. Processor 100 may then control ultrasound module 112 to deliver ultrasound energy to a targeted region (e.g., targeted region 18 or 20) of brain 16 associated with the selected region of brain 16 (126). Processor 100 may then monitor the brain state of the selected region in response to delivering the ultrasound energy (e.g., during delivery or after terminating delivery) (128). If processor 100 has not sensed an elevated brain state (“NO” branch of block 128), processor 100 may adjust one or more parameter values to increase the ultrasound energy (130) and again deliver ultrasound energy with the new parameter values (126).

If processor 100 senses an elevated brain state (“YES” branch of block 128), processor 100 may select the previous ultrasound parameter values as the final ultrasound parameter set (132). The previous ultrasound parameter values may be those values that defined the most recent ultrasound energy that did not cause the elevated brain state. Processor 100 may then store the final ultrasound parameter set in memory 102 for subsequent delivery of neuroprotective ultrasound energy. Processor 100 may also control ultrasound module 112 to deliver neuroprotective ultrasound energy to the targeted region of brain 16 according to the final parameter set (134).

Although the process of FIG. 6 is described as using EEG information to determine brain state, the brain state of patient 12 may be determined using different modalities in other examples. For example, physiological monitoring or imaging sources may include, but are not limited to, reflected ultrasound waves, magnetic resonance imaging (MRI), functional MRI, positron emission tomography (PET), computed tomography (CT), electromyogram (EMG), accelerometer and/or electroencephalogram (EEG). Functional imaging, anatomical imaging, and/or electrophysiological measurements can be used by processor 100 to identify a target region and/or determine brain states of selected regions to determine appropriate ultrasound parameter values for neuroprotective ultrasound energy delivery.

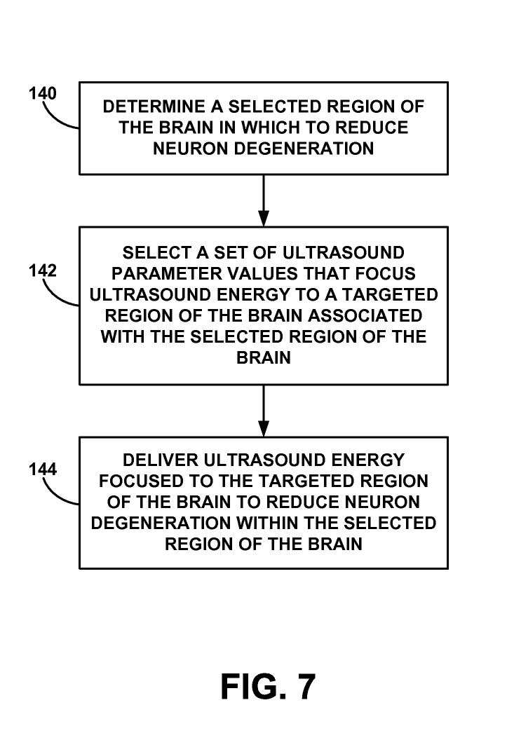

FIG. 7 is a flow diagram that illustrates an example process for delivering ultrasound energy focused to a targeted region of a brain of a patient to reduce neuronal degeneration within a selected region of the brain. As described in FIG. 7, processor 100 of controlling device 28 may be used to deliver neuroprotective ultrasound energy to a targeted region of brain 16. However, in other examples, other devices or systems, such as wearable device 40, may be used to perform such a process.

Processor 100 may determine a selected region of brain 16 in which to reduce neuronal or neuron degeneration (140). Processor 100 may determine the selected region by retrieving instructions from memory 102 or by referencing the diagnosed neurogenerative disease. Processor 100 may then select a set of ultrasound parameter values that focus neuroprotective ultrasound energy to a targeted region of brain 16 associated with the selected region of brain 16 (142). Using the set of ultrasound parameter values, processor 100 may then control ultrasound module 112 to deliver ultrasound energy focused to the targeted region of brain 16 to reduce neuronal degeneration within the selected region of brain 16 (144).

System 10 or wearable device 40 may be configured to reduce the degeneration of neurons caused by Parkinson's disease. For example, neuroprotective ultrasound energy may be relatively low energy ultrasound waves configured to protect dopaminergic neurons in the substantia nigra. This result may be caused by neuron activity protecting the neurons from dopamine depleting neurotoxins. The substantia nigra (SN) may be the selected region and the subthalamic nucleus (STN) may be the targeted region, in one example. Targeted regions and selected regions may include one or both of the SN and STN in another example. In addition, or alternatively, a selected region and/or a targeted region may include the globus pallidus inferior (GPI). For Alzheimer's disease, the selected region and/or targeted region may include the hippocampus, for example.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to system 10, wearable device 40, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques or processes described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

1: A method for reducing or preventing neural degeneration within a brain of a patient, the method comprising:
delivering, via one or more ultrasound transducers, ultrasound energy focused to a targeted region of the brain of the patient according to ultrasound parameters, wherein the ultrasound parameters are selected to generate ultrasound energy that reduces or prevents neural degeneration within at least a portion of the brain.

2: The method of claim 1, wherein the portion of the brain comprises a selected region of the brain, and wherein the method further comprises selecting one or more ultrasound parameter values for the ultrasound parameters that at least partially define delivery of ultrasound energy focused to the targeted region of the brain of the patient that reduces or prevents neural degeneration within the selection region of the brain.

3: The method of claim 2, further comprising:
receiving, by the processor, a first signal from the selected region of the brain;
associating, by the processor, the first signal with a resting state of the selected region of the brain;
selecting, by a processor, an initial ultrasound parameter set;
delivering, by the processor, ultrasound energy to the targeted region of the brain according to ultrasound parameters of the initial ultrasound parameter set;
iteratively adjusting, by the processor, values of one or more of the ultrasound parameters of the initial ultrasound parameter set until the processor receives a second signal from the selected region of the brain indicative of an elevated state of the selected region of the brain; and
selecting, by the processor, the adjusted ultrasound parameters as a final ultrasound parameter set, wherein delivering the ultrasound energy comprises delivering the ultrasound energy according to ultrasound parameters of the final ultrasound parameter set.

4: The method of claim 1, further comprising positioning the one or more ultrasound transducers on an external surface of a head of the patient to focus the ultrasound energy from the ultrasound transducers to the targeted region of the brain of the patient.

5: The method of claim 1, wherein the portion of the brain comprises a selected region of the brain, and wherein the targeted region of the brain comprises at least a part of the selected region of the brain.

6: The method of claim 1, wherein the portion of the brain comprises a selected region of the brain, and wherein the targeted region of the brain comprises first neurons that affect second neurons within the selected region of the brain, the targeted region of the brain being distinct from the selected region of the brain.

7: The method of claim 6, wherein the targeted region of the brain is superficial to the selected region of the brain.

8: The method of claim 1, wherein the one or more ultrasound transducers comprise an array of ultrasound transducers positionable at respective locations on an external surface of a head of the patient.

9: The method of claim 1, wherein the portion of the brain comprises a selected region of the brain, and wherein the ultrasound energy is configured to be below a perception threshold at which the patient perceives delivery of the ultrasound energy and below an activation threshold at which neurons within the selected region of the brain are activated.

10: The method of claim 1, wherein the ultrasound parameters are selected to generate ultrasound energy configured to reduce neural degeneration associated with at least one of Alzheimer's disease, Parkinson's disease, tremor, and dystonia.

11: The method of claim 1, wherein delivering ultrasound energy comprises delivering, from at least two of the one or more ultrasound transducers stereotactically positioned on an external surface of a head of the patient, ultrasound energy focused to the targeted region of the brain of the patient.

12: A system for reducing or preventing neural degeneration within a brain of a patient, the system comprising:
an ultrasound module configured to deliver, via one or more ultrasound transducers, ultrasound energy focused to a targeted region of the brain of the patient; and
a processor configured to control the ultrasound module to deliver the ultrasound energy to the targeted region according to ultrasound parameters, wherein the ultrasound parameters are selected to generate the ultrasound energy that reduces or prevents neural degeneration within at least a portion of the brain.

13: The system of claim 12, wherein the portion of the brain comprises a selected region of the brain, and wherein the processor is configured to select one or more ultrasound parameters values for the ultrasound parameters that at least partially define delivery of ultrasound energy focused to the targeted region of the brain of the patient.

14: The system of claim 13, wherein the processor is configured to:
receive a first signal from the selected region of the brain;
associate the first signal with a resting state of the selected region of the brain;
select an initial ultrasound parameter set;
deliver ultrasound energy to the targeted region of the brain according to the ultrasound parameters of the initial ultrasound parameter set;
iteratively adjust values of one or more of the ultrasound parameters of the initial ultrasound parameter set until the processor receives a second signal from the selected region of the brain indicative of an elevated state of the selected region of the brain; and
select the adjusted ultrasound parameters as a final ultrasound parameter set, wherein the processor controls the ultrasound module to deliver the ultrasound energy according to ultrasound parameters of the final ultrasound parameter set.

15: The system of claim 12, further comprising a wearable head device comprising a housing and the one or more ultrasound transducers mounted within the housing, wherein the wearable head device is configured to position the one or more ultrasound transducers on an external surface of a head of the patient to focus the ultrasound energy from the ultrasound transducers to the targeted region of the brain of the patient.

16: The system of claim 12, wherein the portion of the brain comprises a selected region of the brain, and wherein the targeted region of the brain comprises at least a portion of the selected region of the brain.

17: The system of claim 12, wherein the portion of the brain comprises a selected region of the brain, and wherein the targeted region of the brain comprises first neurons that affect second neurons within the selected region of the brain, the targeted region of the brain being distinct from the selected region of the brain.

18: The system of claim 12, wherein the portion of the brain comprises a selected region of the brain, and wherein the ultrasound module is configured to deliver the ultrasound energy below a perception threshold at which the patient perceives delivery of the ultrasound energy and below an activation threshold at which neurons within the selected region of the brain are activated.

19: The system of claim 12, wherein the ultrasound parameters are selected to generate ultrasound energy configured to reduce neural degeneration associated with at least one of Alzheimer's disease, Parkinson's disease, tremor, and dystonia.

20: A system for reducing or preventing neural degeneration within a brain of a patient, the system comprising:
means for delivering ultrasound energy focused to a targeted region of the brain of the patient; and
means for controlling the means for delivering ultrasound energy to deliver the ultrasound energy to the targeted region according to ultrasound parameters, wherein the ultrasound parameters are selected to generate the ultrasound energy that reduces or prevents neural degeneration within at least a portion of the brain.

21: The system of claim 20, further comprising means for positioning one or more ultrasound transducers on an external surface of a head of the patient to focus the ultrasound energy from the ultrasound transducers to the targeted region of the brain of the patient.
